(19) United States
(12) Patent Application Publication
(10) Pub. No.: US 2024/0009207 A1
(43) Pub. Date: Jan. 11, 2024

(54) CANNABIDIOL (CBD) BASED COMPOSITION AND METHOD FOR TREATING PAIN

(71) Applicant: India Globalization Capital, Inc., Potomac, MD (US)

(72) Inventors: Ramachandra MUKUNDA, Potomac, MD (US); Jagadeesh Sridhara RAO, Ashburn, VA (US)

(73) Assignee: India Globalization Capital, Inc., Potomac, MD (US)

(21) Appl. No.: 18/005,819

(22) PCT Filed: Jul. 19, 2021
(86) PCT No.: PCT/US2021/042243

§ 371 Date: Jan. 17, 2023

Related U.S. Application Data
(60) Provisional application No. 63/053,558, filed on Jul. 17, 2020.

(51) Int. Cl. A61K 31/00; A61K 31/125; A61K 36/886; A61K 36/67; A61K 31/05; A61K 31/417; A61K 31/60; A61K 31/375; A61K 31/165; A61K 31/355; A61K 47/32; A61K 47/10; A61K 47/18; A61P 23/02; A61K 9/70; A61K 9/06

(52) U.S. Cl. CPC A61K 31/658; A61K 31/125; A61K 36/886; A61K 36/67; A61K 31/05; A61K 31/417; A61K 31/60; A61K 31/375; A61K 31/165; A61K 31/355; A61K 47/32; A61K 47/10; A61K 47/18; A61P 23/02; A61K 9/7023; A61K 9/06

(57) ABSTRACT

Compositions and methods for relieving acute pain, muscular pain, neuropathic pain, and the chronic pain by administering an effective amount of an analgesic composition (preferably formulated as a cream, a gel, a lotion, an ointment, or a salve) including at least: (i) one or more cannabis compounds; (ii) histamine dihydrochloride, salicylate, camphor, menthol, primrose oil, capsaicin, or any of the combination thereof; and optionally, four or more compounds acting as at least antimicrobial preservative(s), antioxidant(s), dispersant(s), emollient(s), emulsifier(s), humectant(s), lubricant(s), penetration enhancer(s), skin conditioner(s), skin protectant(s), thickener(s), or any of the combination thereof to an affected area of a subject in need of pain relief. In a preferred embodiment, the optional third category contributes six or more compounds. Relief of acute and chronic pain without significant deleterious side effects is provided thereby.

### CROSS-REFERENCE TO RELATED APPLICATION

This application claims benefit of U.S. Provisional Patent Application, Ser. No. 63/053,558, filed Jul. 17, 2020; the entire content of which is incorporated by reference herein.

### FIELD OF INVENTION

This invention relates to compositions and methods for treating chronic and acute pain with cannabis compounds. The invention is useful for relieving chronic and acute pain including muscle strain, backache, cramping, sprained ligament, neuropathic pain, fibromyalgia, osteoarthritis, rheumatoid arthritis, multiple sclerosis, and dysmenorrhea among other types of pain in humans and veterinary animals.

### BACKGROUND OF INVENTION

Chronic pain, one of the most common grounds for adults seeking medical care, has been linked to limitations in mobility and daily activities, dependence on opioids, anxiety and depression, and diminished quality of life (Chappert S M, Burt C W. Ambulatory care visits to physician offices, hospital outpatient departments, and emergency departments: United States, 2001-02. Vital Health Stat. 2006 February; 13(159): 1-66; Gureje O, Von Korff M, Simon G E, Gater R. Persistent pain and well-being: A World Health Organization study in primary care. JAMA. 1998 July; 280(2): 147-51). Population-based estimates of chronic pain among U.S. adults range from 11% to 40% with considerable population subgroup variation. A recent survey indicated that nearly 20% of U.S. adults suffer from chronic pain. Among them, a higher prevalence of chronic pain was reported among women and older adults. Higher incidence of pain was associated with advancing age. Further, it was observed that non-Hispanic, white adults had a significantly higher age-adjusted prevalence of chronic pain compared to all other racial and ethnic subgroups (Dahlhamer J, Lucas J, Zelaya C, Nahin R, Mackey S, DeBar L, Kerns R, Von Korff M, Porter L, Helmick C. Prevalence of chronic pain and high impact chronic pain among adults—United States, 2016. MMWR. 2018 September; 67(36): 1001-06).

Chronic pain contributes to an estimated $560 billion each year in direct medical cost, lost productivity, and disability programs (Institute of Medicine. Relieving pain in America: A blueprint for transforming prevention, care, education, and research. National Academies Press. 2011). The American Pain Society recommends that pain be made more visible and categorized as the fifth vital sign. Terminal illnesses are often accom-panied by unbearable pain that is severe and difficult to treat.

Physicians are often reluctant to administer large doses of analgesic drugs for fear of respiratory depression or other complications. The same holds true for currently available opioid-based drug therapies which can produce undesirable side effects such as hallucinations, constipation, sedation, nausea, and dysphoria.

Pain management is a great challenge for health care professionals as pain often can debilitate individuals in ways that affects their day-to-day functioning and productivity. Arthritis, for example, has been particularly problematic for women. Since 1999 there has been a 22% increase in the number of women who attribute their disability to arthritis.

Two types of pain are recognized and associated with different underlying patho-physiological mechanisms. Among them, for generalized pain, touching something too hot, cold, or sharp is concerned with the sensing of a noxious stimulus, which is called nociceptive pain. Nociceptive pain is caused by the activation of the immune system. This inflammatory pain is adaptive, but it still needs to be reduced in patients with ongoing inflammation (e.g., those suffering from rheumatoid arthritis or severe trauma). There is also pain that is not protective but maladaptive, resulting from abnormal functioning of the nervous system. This neuropathic pain, which is not a symptom of disease but rather is itself a disease process of the nervous system, and it can occur after damage to the nervous system (Woolf C J. What is this thing called pain? J Clin Invest. 2010 November; 120(11): 3742-44).

The endocannabinoid system (ECS) consists of two types of endogenous G protein-coupled cannabinoid receptors (CB1 and CB2) located in the mammalian brain and throughout the central and peripheral nervous systems, including tissues associated with the immune system. CB1 and CB2 receptors can also coexist in varied levels of expression in the same locations. Among endogenous cannabinoids, N-arachidonoyl ethanolamine (anandamide), and 2-arachidonoylglycerol (2-AG) have been identified. Among the naturally-occurring phytocannabinoids, cannabidiol (CBD) and delta-9-tetrahydrocannabinol (Δ9-THC) are the most studied from the Cannabis sativa subspecies, which includes hemp and marijuana. Unlike the psychoactive properties associated with Δ9-THC, CBD is non-psychoactive and has also been shown to have low toxicity in humans and other species. Ingested and absorbed CBD is rapidly distributed, and due to its lipophilic nature can easily pass the blood-brain barrier (Maroon J, Bost, J. Review of the neurological benefits of phytocannabinoids. Surg Neurol Intl. 2018 April; 9: 91).

The ECS has been implicated as having a role in mitigating neuropathological pain, anxiety, generalized pain, and chronic pain. CBD is highly lipophilic. It is extracted from plant material, then further processed into an isolate or as a distillate in oil form. CBD is commonly administered orally and is metabolized by liver enzymes CYP3A4 and CYP2C19, which are the major isoforms mediating the metabolism of CBD (Jiang R, Yamaori S, Takeda S, Yamamoto I, Watanabe K. Identification of cytochrome P450 enzymes responsible for metabolism of cannabidiol by human liver microsomes. Life Sci. 2011 August; 89(5-6): 165-70). The bioavailability of CBD is affected by both poor solubility (i.e., low absorption) and the large first-pass effect. Self-micro- or self-nano-emulsifying (NE) drug delivery systems can improve the bioavailability of highly lipophilic compounds (Yen C C, Chen Y C, Wu M T, Wang C C, Wu Y T. Nanoemulsion as a strategy for improving the oral bioavailability and anti-inflammatory activity of andrographolide. Intl J Nanomedicine. 2018 January; 13: 669-80; Gupta S, Kesarla R, Omri A. Formulation strategies to improve the bioavailability of poorly absorbed drugs with special emphasis on self-emulsifying systems. ISRN Pharm. 2013 December; 2013: 848043). An NE formulation comprises ternary phases: oil, water, and surfactant (emulsifying agent). It is a thermodynamically-stable system used as a vehicle to deliver highly lipophilic drugs (Suzuki T. J Soc Cosmet Chem Jpn. 2010 March; 44(2): 103-17).

Studies have shown that an ultra-low dose of THC exhibits an anti-inflammatory property (Rock E M, Limebeer C L, Parker L A. Effect of cannabidiolic acid and Δ9-tetrahydrocannabinol on carrageenan-induced hyperalgesia and edema in a rodent model of inflammatory pain. Psychopharmacology (Berl). 2018 November; 235(11): 3259-71). CBD protects against inflammation induced by pain (Philpot H T, O'Brien M, McDougall J J. Attenuation of early phase inflammation by CBD prevents pain and nerve damage in rat osteoarthritis. Pain. 2017 December; 158(12): 2442-51). However, the synergetic effects of cannabinoids in combination with other analgesic agents has not been studied for chronic pain conditions.

An experimental human trial in chronic pain patients with fibromyalgia shows the complex behavior of inhaled cannabinoids with a partial analgesic response after a single inhalation (van de Donk T, Niesters M, Kowal M A, Olofsen E, Dahan A, van Velzen M.

An experimental randomized study on the analgesic effects of pharmaceutical-grade cannabis in chronic pain patients with fibromyalgia. Pain. 2019 April; 160(4): 860-69).

Studies have indicated that treatment with CBD alone does not mitigate chronic pain conditions. There is a need for combining cannabis compounds with analgesic agents to obtain a synergetic effect mitigating chronic pain. Here, this novel analgesic composition is used to treat chronic and/or acute pain in a subject in need of treatment (e.g., pain relief).

### SUMMARY OF INVENTION

In a first embodiment, an analgesic composition and treatment method are provided for treating intrinsic and extrinsic pain by administering to a patient a composition comprising: (i) a first (cannabis) component comprising one or more selected from cannabidiol (CBD), tetrahydrocannabinol (THC), CBD+THC, CBD alone, hemp oil, cannabis oil, CBD isolate, nano CBD, or any of the combinations thereof (ii) a second (analgesic) component comprising one or more selected from histamine dihydrochloride, a salicylate, camphor, menthol, primrose oil, capsaicin or any of the combinations thereof; and (iii) a third (excipient) component comprising one or more selected from aloe vera, black pepper essential oil, vitamin E, vitamin C, glycerin, witch hazel water, a carbomer, triethanolamine, phenoxyethanol, fragrance, or any of the combinations thereof in an alcohol and/or water (i.e., at least an alcohol, water, or an aqueous alcohol).

In a second embodiment, an analgesic composition and treatment method are provided for treating intrinsic and extrinsic pain by administering to a patient a composition comprising: (i) a first (cannabis) component comprising one or more selected from cannabidiol (CBD), tetrahydrocannabinol (THC), CBD+THC, CBD alone, hemp oil, cannabis oil, CBD isolate, nano CBD, or any of the combinations thereof; (ii) a second (analgesic) component comprising one or more selected from histamine dihydrochloride, a salicylate, camphor, menthol, primrose oil, capsaicin or any of the combinations thereof; and (iii) a third (excipient) component comprising one or more selected from aloe vera, black pepper essential oil, vitamin E, isopropyl palmitate, a propylene glycol, caprylic/capric triglyceride, glyceryl stearate, a ceteareth, dimethicone, octyldodecanol, lecithin, ethylhexylglycerin, phenoxyethanol, medium chain triglyceride (MCT) oil, fragrance, or any of the combinations thereof in an alcohol and/or water (i.e., at least an alcohol, water, or an aqueous alcohol).

CBD may be provided as synthetic CBD, CBD isolate, broad spectrum CBD, nano CBD, or organic CBD. Other cannabis compounds, include, but are not limited to, THC, cannabigerol (CBG), and cannabinol (CBN), which may be provided in synthetic, isolated, or nano form. They may also be solubilized using a nanoemulsion (e.g., nano CBD and nano THC) before being incorporated into the composition.

A preferred first component for either embodiment is CBD (such as synthetic CBD, CBD isolate, broad spectrum CBD, nano CBD, or organic CBD) together with THC (such as synthetic THC, THC isolate, or nano THC). A preferred second component for either embodiment is selected from any one of the analgesic compounds alone; histamine dihydrochloride and methyl salicylate; histamine dihydrochloride, methyl salicylate, camphor, and menthol; histamine dihydrochloride, camphor, and menthol; methyl salicylate, camphor, and menthol; camphor and menthol; camphor and capsaicin; menthol and primrose oil; or menthol and capsaicin, among others. The second component may also have anti-inflammatory activity as well as other immunomodulatory activities.

The analgesic composition may be applied topically to the affected area of a subject suffering from acute or chronic pain. It can be formulated as a cream, a gel, a lotion, an ointment, or a salve, among others. Penetration of the skin by active components may be enhanced by incorporating the analgesic composition in a medical device, such as a patch or plaster, applied over the affected area (e.g., a transdermal patch covered by an occlusive dressing).

Preferred ranges in weight percentage are 2% to 10% CBD, 0.1% to 0.3% THC, 0.05% to 1% histamine dihydrochloride, 1% to 10% salicylate, 0.05% to 5% camphor, 0.05% to 5% menthol, 0.05% to 5% primrose oil, and 0.1% to 1.0% capsaicin. Other preferred ranges in weight percentage are 0.5% to 10% aloe vera, 0.025% to 1% black pepper essential oil, 1% to 5% vitamin E, 1% to 2% vitamin C, 0.1% to 1% witch hazel water, 0.5% to 10% glycerin, 0.5% to 2% isopropyl palmitate, 0.1% to 1% propylene glycol, 0.1% to 2% caprylic/capric triglyceride, 0.1% to 2% glyceryl stearate, 0.1% to 2% ceteareth, 0.1% to 1% dimethicone, 0.1% to 1% octyldodecanol, 0.1% to 1% lecithin, 0.1% to 2% ethylhexylglycerin, 0.5% to 2% carbomer, 0.1% to 2% triethanolamine, 0.5% to 1% phenoxyethanol, and 0.1% to 5% fragrance.

A method of treatment is provided comprising at least a step of administering either of the analgesic compositions to a subject in need of treatment at an affected site. As used herein, the term “subject” may be at least a human (male, female, or both) and/or a veterinary animal.

### DETAILED DESCRIPTION OF SPECIFIC EMBODIMENTS

Cannabis compounds can be synthetic (chemically synthesized) or extracted from the seeds and/or flowers of plants such as Cannabis sativa, Cannabis indica, hemp, and hybrid strains of Cannabis sativa and Cannabis indica. Depending on the natural source, the cannabis extract may contain phytocannabinoids such as cannabidiol (CBD), tetrahydrocannabinol (THC), and derivatives thereof; other phytocannabinoids such as cannabinol (CBN), cannabichromene (CBC), and cannabigerol (CBG) among others; terpenoids, which are isoprene polymers in which some methyl groups are moved or removed and/or one or more oxygen atoms are added; and flavonoids, which are polyphenols having a 15-carbon skeleton (i.e., one heterocyclic ring between two phenyl rings). Suitable cannabis compounds are standardized cannabis extract (mostly THC, CBD and CBN) and organic CBD (CBD extracted from cannabis with small or trace amounts of other cannabinoids, terpenoids, and flavonoids).

Artificial (pure) CBD made by chemical synthesis and CBD purified from a natural source (CBD isolate) in crystalline solid or powder form, which are free of THC and other phytocannabinoids, are other suitable cannabis compounds. CBD isolate is a crystalline solid or powder that contains purified CBD. Another source of CBD essentially free of THC is the CBD mixture obtained from hemp or by extracting hemp oil. See Leizer C, Ribnicky0 D, Poulev A, Dushenkov S, Raskin I. The composition of hemp seed oil and its potential as an important source of nutrition. J Nutraceu Funct Med Foods. 2000; 2(4): 35-53. Elixinol CBD (D&G Health, Rye Brooke, New York) is a predominantly CBD product extracted from hemp oil that contains a trace amount of THC. Alternatively, the cannabis compound may be nano CBD, which is any of the aforementioned kinds of CBD in nanoemulsion form.

THC and CBD can be extracted from a Cannabis indica dominant strain using, for example, a 1500-20 L subcritical/supercritical C02 system made by Apeks Supercritical (Johnstown, Ohio) for high pressure oil extraction with ethanol as a solvent.

A method is provided for treating acute and chronic pain, such as in muscle strain, backache, cramping, sprained ligament, neuropathic pain, fibromyalgia, osteoarthritis, rheumatoid arthritis, multiple sclerosis, and dysmenorrhea by administrating to a subject in need thereof a composition including: (i) an effective amount of cannabis compound(s), such as CBD, THC, hemp oil, cannabis oil, or any combination thereof; (ii) an effective amount of analgesic compound(s), such as histamine dihydrochloride, a salicylate, camphor, menthol, primrose oil, capsaicin, or any combination thereof; and (iii) other compounds selected from aloe vera, black pepper essential oil, vitamin E, vitamin C, witch hazel water, glycerin, isopropyl palmitate, caprylic/capric triglyceride, a propylene glycol, cetearyl alcohol, glyceryl stearate, a ceteareth, a PEG stearate, dimethicone, octyldodecanol, lecithin, ethylhexylglycerin, a carbomer, phenoxyethanol, triethanolamine, MCT oil, and fragrance that are dissolved in a vehicle of ethyl alcohol and/or water (i.e., at least ethyl alcohol, water, or aqueous ethanol).

A common mechanism for chronic pain is related to the inflammatory process. The cannabinoids present in CBD oil interact with the endocannabinoid system through the two major cannabinoid receptors: CB1 and CB2. They are the receptors that are found on immune and brain cells throughout the body. Like CBD, another cannabinoid compound THC in combination with CBD has been found to be beneficial in chronic inflammation, such as in multiple sclerosis patients, with limited deleterious side effects.

At least one other compound in the claimed composition serves as a synergetic agent for boosting the cannabis compound's analgesic effect. The synergetic agents include histamine dihydrochloride, a salicylate, camphor, menthol, primrose oil, and capsaicin. Histamine dihydrochloride's effect is due to vasodilation, while salicylate's effect is caused by increasing blood flow. Diclofenac and ketoprofen are alternative topical analgesics; each or both may be used instead of or in addition to histamine dihydrochloride. Camphor and menthol produce a cooling sensation. Primrose oil contains linoleic acid and gamma-linolenic acid. Capsaicin activates its receptor TRPV1, excites sensory neurons, and desensitizes somatic nociceptive fibers.

Histamine is a biological mediator that plays an important role in physiological and pathological processes. It performs an important role in inflammation, immunity, gastric acid secretion, and smooth muscle contraction, and as a neurotransmitter. Histamine is produced and released by different human cells, especially basophils, mast cells, platelets, histaminergic neurons, lymphocytes, and enterochromaffin cells. Histamine can be provided as histamine dihydrochloride; both of which can be used as topical analgesics (Criado P R, Criado R F J, Maruta C W, Machado Filho C A. Histamine, histamine receptors and antihistamines: New concepts. An Bras Dermatol. 2010; 85(2): 195-210).

Methyl salicylate is hydrolyzed to salicylate, which has anti-inflammatory and analgesic effects. In addition, it irreversibly inhibits platelet aggregation and increases local blood flow and the temperature of tissue (Ohta T, Imagawa T, Ito S. Involvement of transient receptor potential vanilloid subtype 1 in analgesic action of methylsalicylate. Mol Pharmacol. 2009 February; 75(2): 307-17; Davis R E, Wachholz J H, Jassir D, Perlyn C A, Agrama M H. Comparison of topical anti-ischemic agents in the salvage of failing random-pattern skin flaps in rats. Arch Facial Plast Surg. 1999 January-March; 1(1): 27-32). An alternative is trolamine salicylate.

Extracted from the wood of camphor trees, camphor oil has been used to relieve pain and inflammation in joints and muscles (Hamidpour R, Hamidpour S, Hamidpour M, Shahlari M. Camphor (Cinnamomum camphora), a traditional remedy with the history of treating several diseases. Intl J Case Rep Imag. 2013; 4: 86-89). Camphor possesses anti-oxidative and anti-inflammatory properties, possibly by blocking production of cytokines and other mediators of inflammation and modulating oxidative stress (Lee H J, Hyun E A, Yoon W J, Kim B H, Rhee M H, Kang H K, Cho J Y, Yoo E S. In vitro anti-inflammatory and anti-oxidative effects of Cinnamomum camphora extracts. J Ethnopharmacol. 2006 February; 103(2): 208-16).

Menthol isolated from menthol oil has been used as a pain reliever and has an analgesic effect in various animal models of nociceptive pain (Atta A H, Alkofahi A. Anti-nociceptive and anti-inflammatory effects of some Jordanian medicinal plant extracts. J Ethnopharmacol. 1998 March; 60(2): 117-24).

Primrose oil is extracted from the seeds of Oenothera biennis. It contains two different omega-6-fatty acids: linoleic acid (about 70%-80%) and gamma-linoleic acid (about 8%-15%). They are not synthesized in the body and are considered essential oils. Primrose oil also contains other fatty acids, aliphatic alcohols, sterols, and polyphenols. Capsaicin (trans-8-methyl-N-vanillyl-6-nonenamide) was isolated from pepper plants of the genus Capsicum, family Solanaceae. Capsaicin may act by binding to the transient receptor potential vanilloid 1 (TRPV1). Topical capsaicin medications are used for the treatment of painful states derived from neuralgia, diabetic neuropathy, osteoarthritis and rheumatoid arthritis. Capsaicin-based preparations are also in use to treat pain due to pruritus, psoriasis, mastectomy, and bladder disorders.

Aloe vera (aloe vera gel) has been used in the treatment of a variety of disorders including wounds and burns. Emodin is an active component of aloe vera and exerts an anti-inflammatory effect by suppressing activation of NF-κB and expression of cell surface adhesion proteins in cells (Chithra P, Sajithlal G B, Chandrakasan G. Influence of aloe vera on the glycosaminoglycans in the matrix of healing dermal wounds in rats. J Ethnopharmacol. 1998 January; 59(3): 179-86; Kumar A, Dhawan S, Aggarwal B B. Emodin (3-methyl-1,6,8-trihydroxyanthraquinone) inhibits TNF-induced NF-κB activation, IκB degradation, and expression of cell surface adhesion proteins in human vascular endothelial cells. Oncogene. 1998 September; 17(7): 913-18).

Black pepper essential oil can act as an anti-inflammatory agent and has been shown to modulate biologic processes like tissue remodeling and wound healing (Han X, Beaumont C, Rodriguez D, Bahr T. Black pepper (Piper nigrum) essential oil demonstrates tissue remodeling and metabolism modulating potential in human cells. Phytother Res. 2018 September; 32(9): 1848-52).

Vitamin E (tocopherol) is a strong antioxidant having several applications in cosmetics and drug delivery systems. It may be provided as D-α-tocopheryl polyethylene glycol-1000 succinate (TPGS-1000). One thousand is the average molecular weight of the polyethylene glycol (PEG) portion of TPGS-1000, which can be produced by esterification of vitamin E succinate with PEG-1000. The biological and physicochemical properties of TPGS provide multiple advantages for its use in drug delivery like high biocompatibility, enhancement of drug solubility, and improvement of drug permeation (Yang C, Wu T, Qi Y, Zhang Z. Recent advances in the application of vitamin E TPGS for drug delivery. Theranostics. 2018 January; 8(2): 464-85).

Vitamin C (ascorbate) is also a potent antioxidant having several applications in cosmetics and drug delivery systems. It can stabilize active compounds in the analgesic composition and improve delivery to their site of action (see Carita A C, Fonseca-Santos B, Shultz J D, Michniak-Kohn B, Chorilli M, Leonardi G R. Vitamin C: One compound, several uses. Advances for delivery, efficiency and stability. Nanomedicine. 2020 February; 24: 102117).

Witch hazel water is distilled from an extract of the leaves and woody parts of a shrub (Hamamelis virginiana). Like aloe vera, witch hazel water can be used as a skin conditioner to tone (tighten) the skin.

Glycerin may act as an emollient and/or. a humectant It can play multiple other roles, acting as an emulsifier, a thickener, or a solvent. Isopropyl palmitate may act as an emollient. Caprylic/capric triglyceride is a mixed ester of caprylic and capric fatty acids attached to a glycerin backbone. It can act as an antioxidant and/or an emollient.

Carbomers (polyacrylates) are acrylic acid polymers, typically crosslinked with allyl ethers of polyalcohols. A carbomer may be provided as carbomer-940 or carbomer-980.

After the carbomer is dispersed in an aqueous solution, neutralizing its carboxylate groups by adding a basic compound (e.g., triethanolamine) to the dispersion can ionize the carbomer and cause its volume to swell. Such carbomers can be used to formulate the analgesic composition as a gel. Alternatively or additionally, the carbomer can be used as a thickener to adjust the viscosity or as a stabilizer of an emulsion.

Triethanolamine is another emulsifier that can also thicken the emulsion. Further, it can neutralize an acidic solution and affect the properties of other compounds in a composition. For example, after becoming hydrated, a carbomer can ionize its polymer chains and cause them to uncoil, thereby increasing the solution's viscosity. Ultimately, this may cause gelation of the solution (e.g., an analgesic composition forming a gel).

Cetearyl alcohol is a combination of cetyl alcohol and stearyl alcohol. A mixture of cetearyl alcohol and glyceryl stearate may form an emulsifying blend to make an oil-in-water emulsion out of the analgesic composition.

Ceteareths are polyethylene ethers of a mixture of saturated fatty alcohols. A preferred compound is ceteareth-20, which has an average number of about 12 monomers of ethylene oxide in the polyethylene chain. They can be used as emulsifiers.

Dimethicone (polydimethylsiloxane) has the desirable property of feeling slippery without feeling oily. Further, it can act as an emollient.

Ethylhexylglycerin has an ethylhexyl group linked to glycerin by an ether bond. It can act as an emollient and a humectant, as well as enhancing the antimicrobial effect of some preservatives.

Lecithin is a mixture of the diglycerides of stearic, palmitic, and oleic acids, linked to the choline ester of phosphoric acid. It can be used as an emollient and/or an emulsifier. Octyldodecanol (2-octyldodecanol) can also be used as an emollient and/or an emulsifier. Polyethylene glycol (PEG) stearates are water soluble esters of PEG and stearic acid. A preferred compound is PEG-100 stearate, which has an average number of about 100 monomers of ethylene oxide in the polyethylene chain. It can act as an emollient, a skin conditioner (moisturizer), and/or an emulsifier, especially in creams and lotions.

Phenoxyethanol (2-phenoxyethanol) has a wide spectrum of antimicrobial activity and has been extensively used as a preservative in cosmetics (Dréno B, Zuberbier T, Gelmetti C, Gontjo G, Marinovich M. Safety review of phenoxyethanol when used as a preservative in cosmetics. J Eur Acad Dermatol Venereol. 2019 November; 33 (suppl. 7): 15-24)). It is effective against various Gram-negative and Gram-positive bacteria, as well as against yeasts. It has been shown to have a weak inhibitory effect on resident skin flora. Medium chain triglyceride (MCT) oil can be used as a penetration enhancer. Triglycerides consist of a glycerol molecule and three fatty acids. Medium chain triglycerides have fatty acid chains that are 6-12 carbons long: caproic acid (C6), caprylic acid (C8), capric acid (C10), and lauric acid (C12). MCT oil may contain caprylic acid, capric acid, or any combination of both caprylic acid and capric acid. Caproic acid is not usually included in the MCT oil due to its unpleasant smell. Lauric acid is often intentionally omitted or removed from the MCT oil, or is present in only a small or even trace amount.

Symptoms of chronic pain include lack of energy, lack of motivation, and associated body aches. Chronic pain is often associated with fibromyalgia, arthritis, multiple sclerosis, dysmenorrhea, HIV/AIDS, and other chronic illnesses.

The combination of CBD and THC unexpectedly leads to enhanced anti-inflammatory function and relieves pain without deleterious side effects.

The combination of CBD and histamine dihydrochloride unexpectedly leads to relief from pain; faster recovery time; faster relief from arthritis, neuropathic pain, and sore muscles; and relief from associated pain with minimal deleterious side effects.

The combination of CBD and methyl salicylate unexpectedly leads to relief from pain arising from strains and sprains, neuropathic pain, pain associated with arthritis without significant deleterious side effects.

The combination of CBD with camphor and menthol unexpectedly leads to relief from pain from strains (i.e., tearing or stretching of the muscles or tendons), sprains (i.e., tearing or stretching of the ligaments), and general musculoskeletal soreness with minimal deleterious side effects.

The combination of CBD with capsaicin unexpectedly leads to relief from neuropathic pain and pain associated with arthritis without significant deleterious side effects.

The combination of CBD with menthol and/or primrose oil unexpectedly leads to relief from pain associated with dysmenorrhea without significant deleterious side effects.

This unique combination of CBD or THC or hemp oil, or cannabis oil result in synergetic effect on pain as well as on anti-inflammatory effects. CBD also plays an important role in decreasing inflammation and relieves pain.

The composition may also contain other components such as aloe vera (an aqueous gel) and/or vitamin E (an oil), which facilitate skin penetration by the cannabis compound(s) and relieve skin soreness. Vitamin E and vitamin C can act as preservatives for cannabinoids like CBD and THC, as well as other components, and enhance their stability in the solution.

CBD, THC, hemp oil, and cannabis oil are insoluble in water due to the hydrophobicity of the compounds. Such hydrophobic compounds may be solubilized in water am emulsification process, especially within structures having sizes of about 100 nm to 1000 nm in the oil phase (see, e.g., Heilscher T. Ultrasonic production of nano-size dispersions and emulsions. Dan Eur Nano Sys Workshop-ENS 2005. 138-143), to dissolve them in water or another aqueous solution. This technology involves emulsifying hydrophobic compounds in the aqueous solution by ultrasound cavitation. Several other known techniques are also available to dissolve CBD, THC, hemp oil, and cannabis oil in aqueous solutions.

The analgesic composition can be formulated as a cream, a gel, a lotion, an ointment, or a salve. The solvent may be an alcohol (e.g., ethyl or isopropyl alcohol), purified water, a mixture of miscible liquids (e.g., ethyl alcohol and water mixed as aqueous ethanol), or the like; which is suitable for cosmetics and topical application. A liquid composition can be a dispersion or an emulsion. A solid (or semisolid) form can be made by adding a mixed petroleum distillate (e.g., mineral oil or petrolatum), a malleable wax (e.g., beeswax, lanolin, paraffin wax, or alkyl silicones), or the like; which is suitable for cosmetics and topical application. As an alternative to animal and petroleum products, plant oils, fats, and waxes can be substituted. For example, mixing two or more of coconut oil, palm oil, olive oil, cocoa butter, shea butter, berry wax, bayberry wax, and jojoba esters. These additives to make solid (or semisolid) forms can be used as a base when formulating a salve. The solvent, base, or penetration enhancer might facilitate at least one cannabis compound and at least one analgesic compound to act synergistically in the dermis of a subject. Components may be delivered across the skin by a medical device, such as a patch or plaster, which incorporates the cannabis compound(s), the analgesic compound(s), or both.

From the foregoing, it would be apparent to a person of skill in this art that the disclosure can be embodied in other specific forms without departing from its spirit or essential characteristics. While the disclosure has been described in connection with what is presently considered to be the most practical and preferred embodiment, it is to be understood that the disclosure is not to be limited to the disclosed embodiment, but on the contrary, is intended to cover various modifications and equivalent arrangements included within the spirit and scope of the appended claims.

### EXAMPLES

The Visual Analog Scale (VAS) is a pain rating scale based on self-reported measures of symptoms that are recorded with a single mark placed at one point along the length of a 10-cm line that represents a continuum between the two ends of the scale-“no pain” on the left end (0 cm) of the scale and the “worst pain” on the right end of the scale (10 cm) (Alexander I. Electronic medical records for the orthopaedic practice. Clin Orthop Relat Res. 2007; 457: 114-19). VAS score for a subject before and after treatment is used to evaluate the degree of pain relief.

### Example 1

A combination of 5% or 7.5% CBD isolate or nano CBD (100 mg/gm) and 0.3% THC is mixed in purified water along with 5% camphor, 1% menthol, 2% aloe vera gel, 0.1% black pepper essential oil, 2% TPGS-1000 (vitamin E), 1% vitamin C, 1% witch hazel water, 2% glycerin, 2% carbomer-940 or -980, 1% phenoxyethanol, 0.1% triethanolamine, and 5% fragrance and applied to a subject, suffering pain from multiple sclerosis. Prior to administration of the formulation, the subject scores 8 on the Visual Analogue Scale to evaluate Pain severity (VAS-P). The scale is from 1 to 10 where 10 represents extreme pain. About 30 min after administration of 1 gm of the formulation, the subject scores 6 on the VAS-P scale.

### Example 2

A combination of 2.5% or 7.5% CBD isolate or nano CBD (100 mg/gm) and 0.3% THC is mixed in purified water along with 0.10% histamine dihydrochloride, 5% camphor, 1% menthol, 2% aloe vera gel, 0.1% black pepper essential oil, 2% TPGS-1000 (vitamin E), 1% vitamin C, 1% witch hazel water, 2% glycerin, 2% carbomer-940/980, 1% phenoxyethanol, 0.1% triethanolamine, and 5% fragrance and applied to a subject, suffering pain from arthritis. Prior to administration of the formulation, the subject scores 8 on the Visual Analogue Scale to evaluate Pain severity (VAS-P). The scale is from 1 to 10 where represents extreme pain. About 30 min after administration of 1 gm of the formulation, the subject scores 6 on the VAS-P scale.

### Example 3

A combination of 5% CBD isolate or nano CBD (100 mg/gm) and 0.25% THC is mixed in purified water along with 0.1% histamine dihydrochloride or 10% methyl salicylate or 5% camphor or 2% menthol or 1% capsaicin. In addition to the foregoing, other compounds include 2% aloe vera gel, 0.1% black pepper essential oil, 2% TPGS-1000 (vitamin E), 1% isopropyl palmitate, 2% caprylic/capric triglyceride, 1% propylene glycol, 1% cetearyl alcohol, 1% glyceryl stearate, 1% ceteareth 20, 1% PEG-100 stearate, 1% dimethicone, 1% octyldodecanol, 1% lecithin, 0.5% ethylhexylglycerin, 1% phenoxyethanol, and 5% MCT oil and applied to a subject, who is suffering generally from pain. Prior to administration of the formulation, the subject scores 8 on the Visual Analogue Scale to evaluate Pain severity (VAS-P). The scale is from 1 to 10 where 10 represents extreme pain. About 30 min after administration of 1 gm of the formulation, the subject scores 6 on the VAS-P scale.

### Example 4

A combination of 5% or 7.5% CBD isolate or nano CBD (100 mg/gm) and 0.3% THC is mixed in purified water along with 0.1% methyl salicylate, 2% camphor, 2% menthol, 2% aloe vera gel, 0.1% black pepper essential oil, 2% TPGS-1000 (vitamin E), 1% vitamin C, 1% witch hazel water, 2% glycerin, 2% carbomer-940/980, 1% phenoxyethanol, 0.1% triethanolamine, and 5% fragrance and applied to a subject, suffering from neuropathic pain. Prior to administration of the formulation, the subject scores 8 on the Visual Analogue Scale to evaluate Pain severity (VAS-P). The scale is from 1 to 10 where represents extreme pain. About 30 min after administration of 1 gm of the formulation, the subject scores 5 on the VAS-P scale.

### Example 5

A combination of 5% or 7.5% CBD isolate or nano CBD (100 mg/gm) and 0.3% THC is mixed in purified water along with 3% camphor, 1% menthol, 2% aloe vera gel, 0.1% black pepper essential oil, 1% TPGS-1000 (vitamin E), 1% vitamin C, 1% witch hazel water, 2% glycerin, 1% carbomer-940/980, 1% phenoxyethanol, 0.1% triethanolamine, and 1% fragrance and applied to a subject, suffering pain from muscle strain. Prior to administration of the formulation, the subject scores 8 on the Visual Analogue Scale to evaluate Pain severity (VAS-P). The scale is from 1 to 10 where 10 represents extreme pain. About 30 min after administration of 1 gm of the formulation, the subject scores 5 on the VAS-P scale.

### Example 6

A combination of 2.5% or 7.5% CBD isolate or nano CBD (100 mg/gm) and 0.3% THC is mixed in purified water along with 5% menthol, 0.1% capsaicin, 2% aloe vera gel, 0.1% black pepper essential oil, 1% TPGS-1000 (vitamin E), 1% vitamin C, 1% witch hazel water, 2% glycerin, 1% carbomer 940/980, 1% phenoxyethanol, 0.1% triethanolamine, and 5% fragrance and applied to a subject, suffering pain from muscle strain. Prior to administration of the formulation, the subject scores 8 on the Visual Analogue Scale to evaluate Pain severity (VAS-P). The scale is from 1 to 10 where 10 represents extreme pain. About 30 min after administration of 1 gm of the formulation, the subject scores 5 on the VAS-P scale.

### Example 7

A combination of 5% or 7.5% CBD isolate or nano CBD (100 mg/gm) and 0.3% THC is mixed in purified water along with 3% menthol, 0.1% capsaicin, 2% aloe vera gel, 0.1% black pepper essential oil, 1% TPGS-1000 (vitamin E), 1% vitamin C, 1%, witch hazel water, 2% glycerin, 1% carbomer-940/980, 1% phenoxyethanol, 0.1% triethanolamine, 5% fragrance, and 5% ethyl alcohol and applied to a subject, suffering pain from muscle strain. Prior to administration of the formulation, the subject scores 8 on the Visual Analogue Scale to evaluate Pain severity (VAS-P). The scale is from 1 to 10 where 10 represents extreme pain. About 30 min after administration of 1 gm of the formulation, the subject scores 5 on the VAS-P scale.

### Example 8

A combination of 2.5% or 7.5% CBD isolate or nano CBD (100 mg/gm) is mixed in purified water with 5% menthol or 5% primrose oil or 3% menthol+3% primrose oil as well as 2% aloe vera gel, 1% TPGS-1000 (vitamin E), 1% vitamin C, 1%, witch hazel water, 2% glycerin, 1% carbomer-940/980, 1% phenoxyethanol, 0.1% triethanolamine, and 5% fragrance and applied to a female subject, suffering cramps and pain in her lower abdomen during menses. Prior to administration of the formulation, the subject scores 8 on the Visual Analogue Scale to evaluate Pain severity (VAS-P). The scale is from 1 to where 10 represents extreme pain. About 30 min after administration of 1 gm of the formulation, the subject scores 5 on the VAS-P scale.

It should be understood that when a range is described, all values within the range are also described (e.g., 1 mg to 10 mg also includes every integer value between 1 mg and 10 mg as well as all intermediate ranges such as 2.2 mg to 8.8 mg, 1.5 mg to 5.2 mg, and 3.6 mg to 8.1 mg). The term “about” may refer to the statistical uncertainty associated with a measurement or the variability in a numerical quantity that a person skilled in the art would understand does not affect operation of the disclosed invention or its patentability (e.g., the quantity±10%).

All modifications and substitutions that come within the meaning of the claims and the range of their legal equivalents are to be embraced within their scope. Different scopes of each embodiment or claim are envisioned. It is envisioned that the term “comprising,” may be substituted with the terms “consisting essentially of,” or “consisting of” throughout this disclosure, including the claims, to change the scope for any aspect of the disclosure.

It should be understood that an element described in this specification should not be construed as a limitation of the claimed disclosure unless it is explicitly recited in the claims. Thus, the granted claims are the basis for determining the scope of legal protection instead of a limitation from the specification which is read into the claims. In contradistinction, the prior art is explicitly excluded from the disclosure to the extent of specific embodiments that would anticipate the claimed invention or destroy its novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the present disclosure. Similarly, generalizations of the description are included as part of this disclosure.

1. An analgesic composition, which comprises: (i) a first component comprising one or more cannabis compounds selected from the group consisting of cannabidiol (CBD), tetrahydrocannabinol (THC), hemp oil, cannabis oil, CBD alone, and CBD together with THC; (ii) a second component comprising one or more analgesic compounds selected from the group consisting of histamine dihydrochloride, a salicylate, camphor, menthol, primrose oil, capsaicin, and any combination thereof, and (iii) a third component comprising four or more selected from the group consisting of aloe vera, black pepper essential oil, vitamin E, vitamin C, witch hazel water, glycerin, a carbomer, phenoxyethanol, and triethanolamine.

2. An analgesic composition, which comprises: (i) a first component comprising one or more cannabis compounds selected from the group consisting of cannabidiol (CBD), tetrahydrocannabinol (THC), hemp oil, cannabis oil, CBD alone, and CBD together with THC; (ii) a second component comprising one or more analgesic compounds selected from the group consisting of histamine dihydrochloride, a salicylate, camphor, menthol, primrose oil, capsaicin, and any combination thereof, and (iii) a third component comprising four or more selected from the group consisting of aloe vera, black pepper essential oil, vitamin E, isopropyl palmitate, caprylic/capric triglyceride, a propylene glycol, cetearyl alcohol, glyceryl stearate, a ceteareth, polyethylene glycol stearate, dimethicone, octyldodecanol, lecithin, ethylhexylglycerin, phenoxyethanol, and medium chain triglyceride (MCT) oil.

3. The composition of claim 1, wherein the first component is only nano CBD and THC.

4. The composition of claim 1, wherein the first component comprises CBD that is provided as synthetic CBD or CBD isolate.

5. The composition of claim 1, wherein the first component comprises THC that is provided as synthetic THC or THC isolate.

6. The composition of claim 1, wherein the second component is histamine dihydrochloride alone; methyl salicylate alone; camphor alone; menthol alone; primrose oil alone; capsaicin alone; only histamine dihydrochloride and methyl salicylate; only histamine dihydrochloride, camphor, and menthol; only methyl salicylate, camphor, and menthol; only camphor and menthol; only camphor and capsaicin; only menthol and primrose oil; or only menthol and capsaicin.

7. The composition of claim 6, wherein the histamine dihydrochloride is chemically synthesized.

8. The composition of claim 1, wherein the third component is aloe vera, black pepper essential oil, and phenoxyethanol.

9. The composition of claim 1, wherein the third component is aloe vera, black pepper essential oil, vitamin E, vitamin C, witch hazel water, and phenoxyethanol.

10. The composition of claim 1, which further comprises one or more fragrances, an optional alcohol such as ethyl alcohol, and water.

11. The composition of claim 1, which is formulated for external use as a cream, a gel, a lotion, an ointment, or a salve.

12. The composition of claim 1, which further comprises a patch, such as a transdermal patch covered by an occlusive dressing, to enhance delivery of at least one or more of the cannabis compounds, at least one or more of the analgesic compounds, or at least one of the cannabis compounds and at least one of the analgesic compounds.

13.-14. (canceled)

15. A method for relieving acute and/or chronic pain, which comprises the step of administering the composition of claim 1 to a subject suffering from pain.

16. The method according to claim 15, wherein the first component is only nano CBD and THC.

17. The method according to claim 15, wherein the first component comprises CBD that is provided as synthetic CBD or CBD isolate.

18. The method according to claim 15, wherein the first component comprises THC that is provided as synthetic THC or THC isolate.

19. The method according to claim 15, wherein the second component is histamine dihydrochloride alone; methyl salicylate alone; camphor alone; menthol alone; primrose oil alone; capsaicin alone; only histamine dihydrochloride and methyl salicylate; only histamine dihydrochloride, camphor, and menthol; only methyl salicylate, camphor, and menthol; only camphor and menthol; only camphor and capsaicin; only menthol and primrose oil; or only menthol and capsaicin.

20. The method according to claim 15, wherein the third component is aloe vera, black pepper essential oil, vitamin E, vitamin C, witch hazel water, and phenoxyethanol.

21. The method according to claim 15, wherein the composition is applied topically as a cream, a gel, a lotion, an ointment, or a salve.

22. The method according to claim 15, wherein the composition further comprises a patch, such as a transdermal patch covered by an occlusive dressing, to enhance delivery of at least one or more of the cannabis compounds, at least one or more of the analgesic compounds, or at least one of the cannabis compounds and at least one of the analgesic compound.
